(19) United States
(12) Patent Application Publication
(10) Pub. No.: US 2012/0172951 A1
(43) Pub. Date: Jul. 5, 2012

(54) MASK APPARATUS FOR SKIN BEAUTY CARE USING LIGHT

(75) Inventor: Hyun Beum Choi, Gwangju (KR)

(73) Assignee: LOU T.S CO., Ltd., Gwangju (KR)

(21) Appl. No.: 13/278,866

(22) Filed: Oct. 21, 2011

(30) Foreign Application Priority Data
Oct. 29, 2010 (KR) 10-2010-0106903

(51) Int. Cl. A61N 5/06

(52) U.S. Cl. 607/91

(57) ABSTRACT

Provided is a mask apparatus for skin beauty care using light. The mask apparatus includes a face mask that is configured to be placed on a human face, a light irradiation unit that is configured to irradiate light into a face of a user wearing the face mask, a light irradiation driving unit that drives the light irradiation unit, a controller that controls the light irradiation driving unit according to an operation signal of the operating unit, and a vibration sensor that senses external vibration or impact applied to the face mask, wherein, when a sensing signal instructing the light irradiation unit to be driven in an ON/OFF mode is received from the vibration sensor, the controller controls the light irradiation unit to be activated according to the sensing signal.

### CROSS-REFERENCE TO RELATED APPLICATION

This application claims priority from Korean Patent Application No. 10-2010-0106903 filed on Oct. 29, 2010 in the Korean Intellectual Property Office, the disclosure of which is incorporated herein by reference in its entirety.

### BACKGROUND OF THE INVENTION

1. Field of the Invention

The present invention relates to a mask apparatus for skin beauty care using light, and more particularly, to a mask apparatus for skin beauty care using light, which is configured to irradiate light into a face of a user in a state in which it covers the user's face.

2. Description of the Related Art

Application of cosmetics to a face is widely used to maintain a younger and beautiful looking face through skin beauty care for preventing a facial skin from loosing elasticity. A protecting barrier layer as a protein protecting layer is formed beneath a horny layer of the epidermis. The outer layer of the skin is protected by a protecting barrier that is a thick protein layer formed beneath the horny layer of the skin, and the outer layer is separated from an inner layer of the skin. Thus, even if a skin care product having an excellent effect of skin protection by adding bio molecules to the skin care product is applied to the skin, wastes on the inner layer cannot be removed. In addition, since cosmetic nutrients cannot penetrate deep into the inner layer, the skin protecting effect may be insufficient.

To overcome the problems, various skin beauty care devices using a galvanic ion current, far-infrared radiation, vibration or the like have recently been proposed.

However, the conventional skin beauty care devices have drawbacks in that they should be operated while continuously grasping the same to bring the same into contact with the skin, which is quite inconvenient in operation.

### SUMMARY OF THE INVENTION

The present invention provides a mask apparatus for skin beauty care using light, which offers operating convenience by allowing the light to be irradiated onto a face in a state the mask apparatus is placed on the face.

According to an aspect of the present invention, there is provided a mask apparatus including a face mask that is configured to be placed on a human face, a light irradiation unit that is mounted within the face mask and configured to irradiate light into a face of a user wearing the face mask, a light irradiation driving unit that drives the light irradiation unit to be turned on/off, an operating unit that selectively operates the light irradiation unit to be driven in an ON/OFF mode, a controller that controls the light irradiation driving unit according to an operation signal of the operating unit, and a vibration sensor that senses external vibration or impact applied to the face mask, wherein, when a sensing signal instructing the light irradiation unit to be driven in an ON/OFF mode is received from the vibration sensor, the controller controls the light irradiation unit to be activated according to the sensing signal.

Preferably, the light irradiation unit includes a first light irradiation unit and a second light irradiation unit, the first light irradiation unit having light emitting diodes arrayed at regions around eyes of a user wearing the face mask to allow the light to be irradiated into the eye regions, and the second light irradiation unit having light emitting diodes arrayed at face regions other than the eye regions, and the operating unit is configured to allow the first light irradiation unit to be driven in an ON/OFF mode by user's selection.

More preferably, the mask apparatus further includes a temperature sensor that senses an internal temperature of the face mask, wherein the controller is set to control the light irradiation unit to be deactivated when the temperature sensed by the temperature sensor reaches a first temperature in an ON mode in which the light irradiation unit irradiates light.

In addition, the face mask may further include a display unit mounted inside the face mask, controlled by the controller and providing display information to the user.

The face mask preferably further includes a USB port capable of interfacing information with an external device.

In the mask apparatus for skin beauty care using light according to the present invention, since the light is irradiated onto a face in a state the mask apparatus is placed on the face, operating convenience can be offered without the need of additional operating works.

### BRIEF DESCRIPTION OF THE DRAWINGS

The above and other features and advantages of the present invention will become more apparent by describing in detail preferred embodiments thereof with reference to the attached drawings in which:

FIG. 1 is a side view of a mask apparatus for skin beauty care using light according to the present invention;

FIG. 2 is a front view of the mask apparatus shown in FIG. 1; and

FIG. 3 is a circuit view of a control system of the mask apparatus shown in FIG. 1.

### DETAILED DESCRIPTION OF THE INVENTION

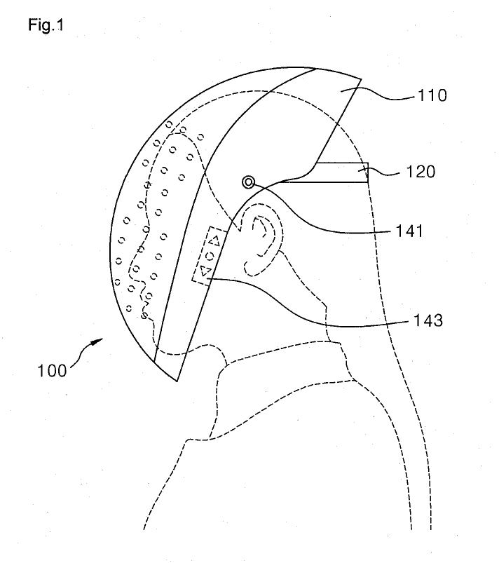

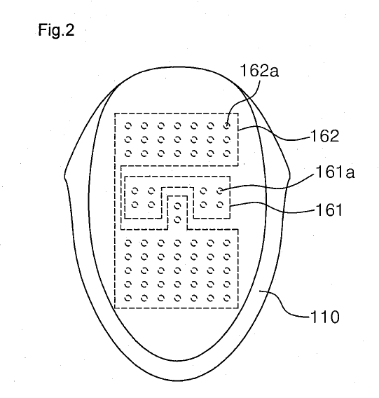

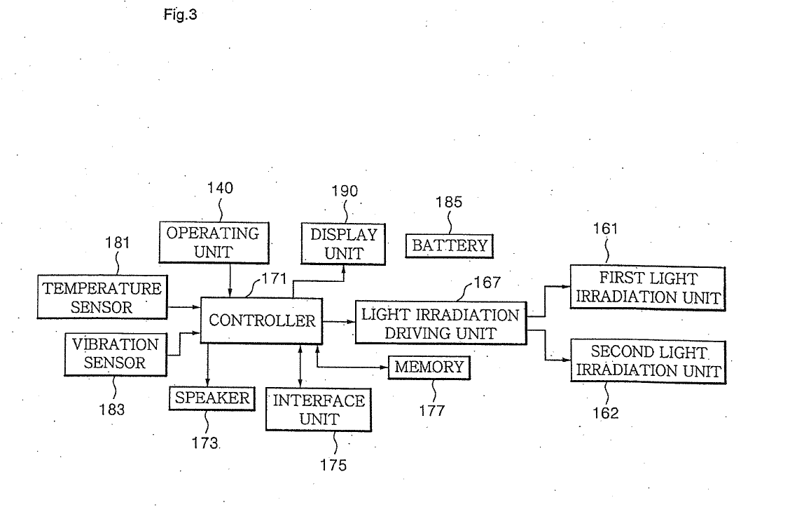

Hereinafter, a mask apparatus for skin beauty care using light according to the present invention will be described in further detail with reference to the accompanying drawings. FIG. 1 is a side view of a mask apparatus for skin beauty care using light according to the present invention, FIG. 2 is a front view of the mask apparatus shown in FIG. 1, and FIG. 3 is a circuit view of a control system of the mask apparatus shown in FIG. 1.

Referring to FIGS. 1 to 3, the mask apparatus 100 for skin beauty care using light includes a face mask 110, first and second light irradiation units 161 and 162, a light irradiation driving unit 167, an operating unit 140, a controller 171, a temperature sensor 181 and a vibration sensor 183.

The face mask 110 is shaped of a hemispherical helmet to be placed on a human face.

The face mask 110 may be made of a hard synthetic resin material.

Reference numeral 120 denotes a tightening band detachably formed to be tightened when the face mask 110 is positioned on the head of a user.

The light irradiation unit is mounted in the face mask 110 and includes a plurality of light emitting diodes (LEDs) 161a and 162a adapted to irradiate light into a face of the user wearing the face mask 110. The light irradiation unit is divided into the first light irradiation unit 161 and the second light irradiation unit 162 so as to selectively control light irradiation according to the portion to be irradiated with light.

The first light irradiation unit 161 has the LEDs 161a arrayed at regions around eyes of a user wearing the face mask 110, to be briefly referred to as ‘eye regions’ hereinafter, to allow the light to be irradiated into the eye regions.

The second light irradiation unit 162 has the LEDs 162a arrayed at face regions other than the eye regions. That is to say, the second light irradiation unit 162 includes the LEDs 162a arrayed at regions excluding the first light irradiation unit 161.

Here, the first light irradiation unit 161 is configured to be independently controlled such that it may be activated in a state in which the user closes his/her eyes, and it may be deactivated by user's selection in a state in which the user opens his/her eyes.

The LED 161a and 162a applied to the first and second light irradiation units 161 and 162 may include infrared LEDs emitting infrared light.

Alternatively, the LED 161a and 162a applied to the first and second light irradiation units 161 and 162 may include LEDs emitting light having a wavelength ranging from 400 nm to 1000 nm.

The light irradiation driving unit 167 is configured to simultaneously or selectively turn the light irradiation unit, that is, the first light irradiation unit 161 and the second light irradiation unit 162.

In addition, the light irradiation driving unit 167 is configured to adjust an average power level by the current applied to the LED 161a and 162a or by varying a duty ratio so as to adjust the intensity of light emitted from the LED 161a and 162a according to the mode supported.

The operating unit 140 may selectively operates the first light irradiation unit 161 and the second light irradiation unit 162 to be driven in an ON/OFF mode. Alternatively, the operating unit 140 may select a mode supported for skin treatment, or other supported functions, such as an operating time change, etc.

In an exemplary embodiment, the operating unit 140 may be configured to support a mode for skin regeneration and wrinkle improvement in which the mask apparatus 110 is used after the skin is massaged with a nutrient cream, or to support a skin sedation mode in which the mask apparatus 110 is used to sedate the skin damaged by ultraviolet light after a sedation pack is applied to the skin. Here, the mode for skin regeneration and wrinkle improvement may be set such that power is ON to activate the light irradiation unit and the light irradiation unit is then turned off after about 15 minutes. The skin sedation mode may be set such that light is irradiated for about 20 minutes by adjusting the intensity of light by repeating a STRONG-WEAK-STRONG-WEAK cycle.

The operating unit 140 is configured to allow the user to selectively turn the first light irradiation unit 161 on or off.

Reference numeral 141 denotes a power ON/OFF switch, and is configured to be activated in cycles in which if the power ON/OFF switch 141 is operated in an OFF state, the first and second light irradiation units 161 162 are both turned on; if the power on/OFF switch 141 is operated once again, the first light irradiation unit 161 is turned off and the second light irradiation unit 162 is turned on; and then if the power ON/OFF switch 141 is operated once more, the first light irradiation unit 161 is turned off and the second light irradiation unit 162 is turned off.

Reference numeral 143 denotes an UP/DOWN key through which the user can set to increase or decrease the light intensity.

Meanwhile, the operating unit 140 may transmit an operation signal wirelessly to the controller 171 to later be described, and the controller 171 may receive the wirelessly transmitted operation signal and process the same.

The temperature sensor 181 is installed in the face mask 110 to sense an internal temperature of the face mask 110 and supplies the sensed temperature to the controller 171.

The display unit 190 is controlled by the controller 171 and displays display information thereon.

The display unit 190 may be configured to display an operating state and the temperature sensed by the temperature sensor 181.

More preferably, the display unit 190 may be configured to display picture information. In this case, the user wearing the mask apparatus 100 in use can fill in time watching a variety of multimedia information recorded in a memory 177 or an interface unit 175, that is, a USB port, which will later be described, through the display unit 190.

The vibration sensor 183 is employed to instruct the light irradiation unit to be turned on or off by allowing the user to manipulate vibration or impact. That is to say, the vibration sensor 183 may be any type of sensor as long as it is capable of sensing external vibration or impact applied to the face mask 110.

For example, the vibration sensor 183 may be a thin-film type piezoelectric sensor provided on a surface of the face mask 110.

The speaker 173 is controlled by the controller 171 and outputs sound information.

The battery 185 supplies power required for driving components of the mask apparatus 100, and is preferably rechargeable battery.

In addition, the face mask 110 may be configured to receive external power through an adapter.

The interface unit 175 is employed to support interfacing with an external device, and is preferably a USB port.

The controller 171 controls the light irradiation driving unit 167 to activate both of the first light irradiation unit 161 and the second light irradiation unit 162 or to activate only the second light irradiation unit 162 to irradiate light according to the operation signal of the operating unit 140.

In addition, when the temperature sensed by the temperature sensor 181 in an ON mode in which at least one of the first light irradiation unit 161 and the second light irradiation unit 162 irradiates light reaches a first temperature, e.g., 40°, the controller 171 determines an internal temperature state of the face mask 110 as being ‘overheated’ and controls the light irradiation unit to be deactivated.

In addition, when a sensing signal instructing the light irradiation unit to be driven on or off is received from the vibration sensor 183, the controller 171 controls the light irradiation unit to be driven according to the sensing signal. In an exemplary embodiment, if the face mask 110 is tapped three times, that is, if a three-times-tapping signal is sensed from the vibration sensor 183, the controller 171 determines that there is a power ON instruction, and controls the light irradiation unit to be activated. If the face mask 110 is tapped two times, that is, if a two-times-tapping signal is sensed from the vibration sensor 183, the controller 171 determines that there is a power OFF instruction, and controls the light irradiation unit to be deactivated.

Meanwhile, when an external memory is connected to a USB port employed as the interface unit 175, the controller 171 supports multimedia files recorded in the external memory to be displayed on the display unit 190 according to the operation signal of the operating unit 140.

While the present invention has been particularly shown and described with reference to exemplary embodiments thereof, it will be understood by those of ordinary skill in the art that various changes in form and details may be made therein without departing from the spirit and scope of the present invention as defined by the following claims. It is therefore desired that the present embodiments be considered in all respects as illustrative and not restrictive, reference being made to the appended claims rather than the foregoing description to indicate the scope of the invention.

What is claimed is:

1. A mask apparatus for skin beauty care using light, the mask apparatus comprising:
a face mask that is configured to be placed on a human face;
a light irradiation unit that is mounted within the face mask and configured to irradiate light into a face of a user wearing the face mask;
a light irradiation driving unit that drives the light irradiation unit to be turned on/off;
an operating unit that selectively operates the light irradiation unit to be driven in an ON/OFF mode;
a controller that controls the light irradiation driving unit according to an operation signal of the operating unit; and
a vibration sensor that senses external vibration or impact applied to the face mask,
wherein, when a sensing signal instructing the light irradiation unit to be driven in an ON/OFF mode is received from the vibration sensor, the controller controls the light irradiation unit to be activated according to the sensing signal.

2. The mask apparatus of claim 1, wherein the light irradiation unit includes a first light irradiation unit and a second light irradiation unit, the first light irradiation unit having light emitting diodes arrayed at regions around eyes of a user wearing the face mask to allow the light to be irradiated into the eye regions, and the second light irradiation unit having light emitting diodes arrayed at face regions other than the eye regions, and the operating unit is configured to allow the first light irradiation unit to be driven in an ON/OFF mode by users selection.

3. The mask apparatus of claim 1, further comprising a temperature sensor that senses an internal temperature of the face mask, wherein the controller is set to control the light irradiation unit to be deactivated when the temperature sensed by the temperature sensor reaches a first temperature in an ON mode in which the light irradiation unit irradiates light.

4. The mask apparatus of claim 3, wherein the face mask further comprises a display unit mounted inside the face mask, controlled by the controller and providing display information to the user.

5. The mask apparatus of claim 3, wherein the face mask further comprises a USB port capable of interfacing information with an external device.
